(19) United States
(12) Patent Application Publication
(10) Pub. No.: US 2005/0118705 A1
(43) Pub. Date: Jun. 2, 2005

(54) Electrical detectors for microanalysis

(76) Inventors: Richard D. Rabbitt, (US); H. Edward Ayliffe, (US)

Correspondence Address: TRASK BRITT, P.O. BOX 2550, SALT LAKE CITY, UT, 84110, US

(21) Appl. No.: 10/983,219

(22) Filed: Nov. 5, 2004

Related U.S. Application Data
(60) Provisional application No. 60/518,094, filed on Nov. 7, 2003.

(51) Int. Cl. C12Q 1/68; H01L 29/08; H01L 35/24; C12M 1/34

(52) U.S. Cl. 435/287.1; 257/40

(57) ABSTRACT

Apparatus and methods for performing microanalysis of particles using a microelectrical-mechanical system (MEMS) chip to electrically interrogate the particles. The MEMS chip is typically manufactured using known lithographic micromachining techniques, employed for example, in the semiconductor industry. A substrate carries a plurality of microelectrodes disposed in a detection zone and spaced apart along an axis of a microchannel. The microchannel is sized in cross-section to cause particles carried by a fluid to move past the electrodes in single file. Impedance is measured between one or more pairs of electrodes to determine the presence of a particle in the detection zone. In certain embodiments used in cell manipulation, an electroporation signal may be applied between one or more pairs of electrodes to enhance permeability of a cell membrane. A structural arrangement may be provided to introduce a treatment substance into the microchannel in the vicinity of a cell, which may be restrained for a period of time in a treatment zone.

### CROSS-REFERENCE TO RELATED APPLICATION

This application claims the benefit of the filing date of U.S. Provisional Patent Application Ser. No. 60/518,094, filed Nov. 7, 2003, for “Electrical Detectors for Microanalysis,” the contents of which are incorporated herein by reference.

### TECHNICAL FIELD

The invention generally relates to detectors for microanalysis of particles carried in a fluid, and methods for making and using such detectors. In one preferred embodiment, the invention relates to detectors for characterizing whole blood based on the electric impedance of the whole blood components and cytometric phenotypes, as well as methods for making and using such detectors.

### BACKGROUND

Electric impedance (EI) is the measure of the degree to which an electric circuit resists electric-current flow when a voltage is applied across its terminals. Impedance, expressed in Ohms, is the ratio of the applied voltage to current. In alternating current (AC) circuits, impedance is a function of the geometry and distribution of dielectric properties (e.g., conductance, permitivity, permeability), and is sometimes modeled using simple circuits including resistance, inductance, and capacitance. Biological solutions, cells, and subcellular structures can be investigated and characterized using EI as the measured parameter. The unique dielectric properties of cells and solutions enable an EI-based detector to identify, for example, relatively small morphological differences between cell types and even molecular changes occurring at electrode-electrolyte interfaces due to specific protein or DNA binding to the surface. Relatively macroscale, whole-cell physical characteristics, such as size and membrane structure, can also be measured using electric impedance-based detection methods.

The Complete Blood Count (CBC) is one of the most common blood tests performed in the United States (˜1.5 billion annually). These tests generally provide clinically relevant information regarding patient health. Unlike other tests performed in the clinical laboratory, the CBC test provides a pathology report on the morphological “appearance” of blood cells and the frequency in which they appear. A CBC test is typically performed for almost every patient with a major illness, in developed countries, because the CBC reveals anemia (low RBC count), leucopenia (low WBC count) and thrombocytopenia (low platelet count) and can indicate viral or bacterial infections.

The more specialized CBC Differential test augments the standard CBC by subdividing the white cells into five “normally appearing” categories (neutrophils, lymphocytes, monocytes, eosinophils, and basophils). Traditionally, CBC Differential tests were manually performed on blood smears and cells were counted based on observed morphological differences. Results were represented as a percentage of the total cells counted (typically, 100 WBCs total). The subjective nature and relatively small total cell count of this technique were major limitations that significantly reduced the reliability of the manual CBC.

These traditional limitations have been the primary drivers for the technological advances leading to state-of-the-art hematology analyzers (employing multiple detectors and chemical reagents). Modern differential cell counters have enhanced diagnostic capabilities compared to standard CBC analyzers and are routinely used for screening, case finding, and monitoring of hematologic and nonhematologic disorders. In practice, the CBC differential count is used to diagnose bacterial or viral infections, evaluate allergic conditions, diagnose and monitor malignant diseases such as leukemia, and staging of HIV infections. Due to the nature of current technology, state of the art hematology analyzers are expensive, physically large, complex units that demand highly skilled and trained technicians for operation.

Electroporation is a well-established technique for generating transient pores in cell membranes via the application of an applied pulsed electric field. Biochemical and genetic studies have routinely use macroscale electropermeabilization of cell membranes in suspension to introduce genes, macromolecules, compounds and drugs into cells. The physical basis for electro-injection of macromolecules has the advantage of being predictable and limiting the side effects typically seen by more conventional methods such as viral transfection, PEG-induced fusion, and chemical/mechanical perturbations of the membrane.

Unfortunately, these state of the art macroscale electroporation techniques expose millions of cells to relatively poorly controlled electroporation pulses and therefore experience extremely poor injection ratios and cell survival ratios. More recently, researches have begun investigating novel methods for single cell electroporation and injection using two opposing carbon fiber electrodes (to apply the necessary transmembrane potential). While this technique is suitable for research applications, it lacks the potential to be used effectively to electro-inject a larger number of cells.

The ability to manipulate membrane potentials of isolated cells and measure their electrical characteristics is a cornerstone of modern cellular electrophysiology. Various methods have been developed to extract the canonical parameters (i.e., membrane capacitance, membrane resistance, and cytoplasmic resistance) of both individual cells and cell aggregates. The electrical properties of cell membranes have traditionally been measured on aggregates of cells using the suspension technique. Single cells have been studied using micropipettes and the whole-cell clamp technique or sucrose gap techniques. The dielectric properties of single cells and particles have also been studied using electrophoresis and electroporation.

Certain devices under development include a sheath fluid, in which a narrow stream of particles carried by a second fluid stream can be urged through an analysis zone, potentially organizing the entrained particles in substantially single file order. Such technology suffers from an inherent spacing between the particles embedded in the sheath fluid and one or more interrogating sensors. The sensor spacing inherent in devices incorporating a fluid sheath can reduce signal strength and cause an attendant loss in sensitivity and accuracy in collected data.

### BRIEF SUMMARY OF THE INVENTION

It would be an advance to provide a sensor capable of urging particles into single-file order for more direct (or close) contact between an interrogating electrode, or array of electrodes, and the particle(s) of interest. The development of microfabricated structures permitting detailed cellular characterization in solutions having ionic strengths similar to physiologic conditions would be a further advance to improve sensitivity and accuracy of dielectric characterization.

The present invention relates generally to detectors for analyzing microscale particles (0.01-100 microns in size) suspended in, or carried by, a fluid. Highly preferred embodiments of the invention find use in characterizing biological solutions and cells using microfabricated electric impedance-based sensors. Currently preferred embodiments of the invention accurately differentiate cells based on morphological, membrane, and/or cytoplasmic characteristics. Such embodiments can be leveraged for use in blood analysis to significantly and dramatically improve the current state of art in hematology analyzers. Microfabricated, EI-based sensors also have application in basic research markets, such as single cell electrophysiology. Certain embodiments of the invention operate as easy to use, inexpensive, disposable sensors for a variety of commercial products including, but not limited to, a micro-CBC analysis system for clinics, a gene therapy system for research and/or treatment of cancer and other disorders, and a research platform for cellular electrophysiology studies.

Devices constructed according to certain principles of the invention improve upon traditional methods for studying single cell electrophysiology in at least one of three aspects: (1) placement of single cells for study; (2) transmembrane electrical study of cells; and (3) ability to regionally control membrane voltages when studying single cell electrophysiology. Certain currently preferred embodiments, having microfabricated architecture, are suitable for flow-through single cell identification (particle counting), and membrane dielectric characterization.

Devices within the ambit of the invention may be manufactured using lithographic micromachining techniques and equipment similar to solid-state component manufacturing equipment commercially established in the semiconductor industry. Such manufacturing capability permits a reduction in cost of components, for example, microelectro-mechanical chips used as a constituent component in cell detection sensors for hematology analyzers. Devices structured according to principles of the invention can be microfabricated into discrete, disposable, simple units capable of performing an equivalent level of hematology analysis to that of current state-of-the-art, large, costly units. Improvements inherent in certain embodiments of the invention provide a reduction in unit cost, pricing, and complexity of operation, while affording a simultaneous and equally significant increase in accessibility and mobility.

Certain embodiments constructed according to the invention include microfabricated architecture that is capable of detecting the presence of a single cell (as it flows through a detection zone) and substantially simultaneously applying a highly uniform electroporation pulse. Such embodiments find exemplary application to electro-injection of macromolecules into the cell. A detection zone and an electroporation zone may overlap, or may be distinct from each other. The application of this technology for electroporation-based macromolecule delivery to isolated cells provides a valuable supplement to traditional technologies and provides a new tool for cancer research and/or treatment of cancer and other diseases using gene therapies.

A first exemplary device within the ambit of the invention is a microelectrical-mechanical system (MEMS) device operable to perform electrically based microanalysis on particles transported in a fluid stream. The MEMS device typically is formed on a substantially electrically non-conductive substrate. The substrate typically supports a machinable layer with a microchannel being formed in the machinable layer. The microchannel is disposed in fluid communication between a receiving chamber and a holding chamber effective to transport the fluid stream for analysis. In general, the microchannel is sized in cross-section directly to effect substantially single-file flow of particles through an interrogation zone having a length. A currently preferred microchannel in a device adapted for analysis of blood-related particles has a cross-section sized about 10 μm by about 10 μm.

A plurality of electrodes are carried on the substrate and disposed in association with the interrogation zone operably to transmit (and/or receive) an electrical signal into/from the microchannel. In certain embodiments, it is preferred to provide a space, between an electrode surface and a boundary wall, which is about 10 μm, to place the cells moving past that electrode into close proximity with, or even to touch the electrode. Desirably, a plurality of remote contact pads are arranged to communicate an electric signal to, or from, cooperating electrodes. Preferred contact pads provide a contact surface sized at least an order of magnitude larger than a surface area of its associated electrode to facilitate connecting the device to a remote electrical signal source. A cover layer is added over the microchannel to form a substantially fluid-tight top to the microchannel.

In certain currently preferred embodiments, a plurality of electrodes are disposed spaced apart along an axis of the microchannel to form an electrical signal therebetween oriented substantially along the channel's axis. Furthermore, most preferred embodiments include electrodes disposed only on one side of the microchannel, to reduce manufacturing cost and complexity. An interrogation zone occupies a portion of the microchannel having a cross-section of substantially uniform size along said length, and a plurality of the electrodes are disposed spaced apart in a direction oriented parallel to said length. Generally, an overall footprint of the MEMS device may be sized about 20 mm by about 20 mm as a trade-off between material cost, manufacturing yield, and ease of handling. In MEMS chips used to analyze blood cells, a cross-section of the microchannel may be sized between about 35 μm² and about 250 μm², or so. Devices having even smaller channels may be used for molecular or nanoparticle detection and interrogation. Certain embodiments include a filter disposed in association with an entrance to the microchannel and arranged to resist passage of clogging particles into the microchannel. It is convenient to form such a filter in the machinable layer.

Embodiments of the invention may also include a fluid delivery conduit disposed to provide fluid communication between a treatment zone in the microchannel and a treatment fluid chamber. Such embodiments may advantageously be used in certain electroporation activities. In some cases, the treatment fluid chamber is carried on the substrate. One device adapted for electroporation includes a first pair or group of electrodes operably arranged to detect the presence of a cell in the microchannel, and a second pair or group of electrodes arranged downstream of at least an individual one of the first pair of electrodes effective to deliver an electroporation signal to a cell in an electroporation treatment zone. Such a device may also include a fluid delivery conduit disposed to provide fluid communication between a treatment fluid chamber and the electroporation treatment zone. Also, an electrode arrangement may be provided in association with the fluid delivery conduit and operable to detect passage of treatment substance therethrough. An additional or alternate electrode arrangement can be provided in association with the fluid delivery conduit and operable to apply an electroporation signal in the vicinity of the electroporation treatment zone. Furthermore, a conduit may be included and disposed to provide fluid communication between the electroporation treatment zone and a vacuum source operable to resist motion of a cell from the electroporation treatment zone.

It is within contemplation to include one or more integrated electrical circuit component carried on the substrate and disposed in-circuit with at least one electrode. Certain embodiments may include a coating applied to wetted surfaces of the microchannel and operable to resist build-up of biologic material in the microchannel. Currently preferred embodiments include electrodes arranged as surface electrodes disposed on a bottom surface of the microchannel, and spaced apart along an axis of the microchannel. In certain cases, the substrate and the cover layer are sufficiently transparent in combination to permit transmission of light therethrough to permit optical surveillance of cell motion through the microchannel.

The invention encompasses various methods for performing microanalysis of particles using a microelectrical-mechanical system (MEMS) chip to electrically interrogate the particles. One such method includes providing a MEMS chip having a microchannel disposed in fluid communication between a receiving chamber and a holding chamber, with the microchannel being structured directly to urge particles into substantially single-file flow past a plurality of electrodes disposed in a detection zone within the microchannel. A fluidized sample (e.g., a mono- or polydisperse suspension of particles or cells in fluid, or collection of particles entrained in the fluid), is loaded into the receiving chamber, and a motive force operable to urge flow of a portion of the sample through the microchannel in a direction toward the holding chamber is applied. As the portion is flowing through the microchannel, a state of electrical impedance is measured between a pair of electrodes to obtain impedance data from the detection zone. Finally, analysis of the impedance data is performed to determine the presence of a particle in the detection zone. It is also within contemplation to extract information regarding electrical properties/signature/spectra of the particle. A method may include performing analysis of the impedance data, including the single frequency, impedance or impedance spectra, or nonlinearities thereof, to determine one or more physical characteristics of certain particles. It is further within contemplation that cells may be sorted based upon the extracted data. A method may also include applying an electroporation signal between a pair of electrodes effective to enhance permeability of a membrane of at least certain particles subsequent to detection of one of the particles in the detection zone. The method may also include applying a restraining force on one of the particles effective to resist movement of that particle from a treatment zone in the microchannel. Furthermore, causing flow of a treatment fluid, including a treatment substance, from a treatment fluid chamber through a conduit disposed in fluid communication with the electroporation treatment zone effective to introduce the treatment substance to the vicinity of the particle can be included as a portion of the method. In some cases, analysis of collected impedance data may be performed to determine an instantaneous flow rate through the microchannel based upon time-of-flight of one or more particles between a first detected position and a second detected position disposed downstream of the first detected position.

### BRIEF DESCRIPTION OF THE SEVERAL VIEWS OF THE DRAWINGS

In the drawings, which illustrate what are currently considered to be the best modes for carrying out the invention:

FIG. 1 is a plan view of the detector in one aspect of the invention.

FIG. 2 illustrates a close-up top view of a detection zone of the detector in one aspect of the invention.

FIG. 3 is side view, partially in section, of a portion of the structure illustrated in FIG. 2, taken through section 3-3, and looking in the direction of the arrows.

FIG. 4 is a plan view of an alternative configuration.

FIG. 5 is a close-up plan view of an operable electrode configuration.

FIG. 6 is a close-up plan view of an alternative electrode configuration.

FIG. 7 is a view in perspective of a microchannel having transversely disposed electrodes.

FIG. 8 is a cross-section view of the structure illustrated in FIG. 7, taken through section 8-8 and looking in the direction of the arrows.

FIG. 9 is a cross-section view of the structure illustrated in FIG. 7, taken through section 9-9 and looking in the direction of the arrows.

FIG. 10 is a view in perspective of a microchannel having axially disposed surface electrodes.

FIG. 11is a cross-section view of the structure illustrated in FIG. 10, taken through section 11-11 and looking in the direction of the arrows.

FIG. 12 is a plan view of an alternative MEMS chip configuration including fluid delivery structure.

FIG. 13 is a close-up of a portion of the structure illustrated in FIG. 12.

FIG. 14is a plan view of an alternative microchannel structured to provide detection electrodes, electroporation electrodes, and fluid delivery.

FIG. 15 is a close-up plan view of a microclamping electrode arrangement.

FIG. 16 illustrates an equivalent electrical circuit configuration operable with certain embodiments structured according to principles of the invention.

FIG. 17 illustrates an electrical circuit configuration operable with certain embodiments structured according to principles of the invention.

FIG. 18 illustrates a method of using the detector in one aspect of the invention.

FIG. 19 is a plot of electrical signals applied and measured across electrodes during the use of one embodiment of the invention.

FIG. 20 is a bar plot showing representative reactance corresponding to several cell types as measured with an embodiment of the invention.

FIG. 21 is a time-based plot showing representative reactance values corresponding to a plurality of cells passing through a detection zone of an embodiment of the invention.

FIG. 22 is a pair of time-based plots showing representative phase and non-dimensional reactance values corresponding to several cell types as measured with an embodiment of the invention.

### DETAILED DESCRIPTION OF THE ILLUSTRATED EMBODIMENTS

The following description provides specific details in order to provide a thorough understanding of the invention. The skilled artisan, however, would understand that the invention can be practiced without employing these specific details. Indeed, the invention can be practiced by modifying the illustrated devices and described methods and can be used in conjunction with certain apparatus and techniques already being conventionally used in industry. It is to be understood that reference to an item being “on” a layer, or carried by a substrate, is intended to encompass the structure as recited and also including one or more intervening layers. Furthermore, structure may be regarded as being carried “by” or being “on” a layer if the structure in question is formed “in” the referenced layer. With respect to material characterization, the word “machinable” is intended to encompass material in which patterns, or structural shapes, can be formed or manipulated using material removal techniques including scratching, cutting, laser ablation, melting, chemical etching, lithographic techniques and processes, and conventional machining operations.

Referring now to FIGS. 1-3, a first exemplary particle detector constructed according to principles of the invention is generally indicated at 100. Detector 100 is structured to operate as a microelectric impedance analyzer, and includes a microelectro-mechanical system (MEMS) chip 102. Chip 102 may be characterized as a microfabricated electric impedance-based flow chip. Chip 102 is capable of communicating electric signals between electrodes from steady-state to radio frequencies, and beyond. In general, chips 102 may be characterized as resembling substantially planar elements, although other form factors are operable. A typical chip 102 may have an overall surface sized about 20 mm by about 20 mm. Structure carried by the chip 102 typically includes tiny (visible with an optical or electron scanning microscope) structures arranged to perform mechanical and electrical operations as a system.

The MEMS chip 102 illustrated in FIGS. 1-3 includes a microchannel 106 structured to provide flow of fluid, and fluid-entrained particles 108, through an interrogation zone generally indicated at 110. A direction of fluid flow along a path through microchannel 106 is indicated generally by arrow 114. Microchannel 106 typically will have a cross-section that is sized in general agreement with a size of particles to be interrogated. Desirably, the cross-section of microchannel 106 will provide structure operable to urge particles 108 for flow in a single-file arrangement along a portion of flow path 114 through microchannel 106.

A currently preferred microchannel in a device adapted for analysis of blood-related particles has a cross-section sized about 10 μm by about 10 μm. Red blood cells are somewhat donut-shaped, and about 7 μm in diameter, and perhaps 2 μm thick. In contrast to devices employing a sheath fluid, or devices employing hydrodynamic focusing, the preferred channel shape encourages the RBCs into very close proximity with the interrogating electrodes, if not into direct contact. Although white blood cells are typically about 12-20 μm in diameter, they have been found to be able to adapt in shape and flow through a microchannel having the preferred smaller size cross-section without sustaining undue cell damage. In certain embodiments, it is preferred for microchannel 106 to be structured to provide a space, between an electrode surface and an opposite boundary wall, which is about 10 μm, or less, to place blood cells moving past that electrode into close proximity with, or even to touch that electrode. Such shallow microchannels may have an increased width to reduce a deformation required of certain cells passing therethrough.

MEMS chip 102 includes a plurality of electrodes 118 disposed to transmit an electrical signal into, across, and/or along microchannel 106. Of course, it is to be realized in general that electrodes are also operable to receive electrical signals as well. A plurality of electrically conductive lead elements 122 are arranged individually to place a selected electrode 118 into electrical communication with a corresponding remote electrical contact pad 126. Pads 126 may be disposed remote from a chip 102 (not illustrated). However, it is currently preferred to include contact pads 126 on board a chip 102 to facilitate making electrical communication with an electrical signal source 130, or other interrogation apparatus. The signal source 130 is typically remote from a chip 102, although such is not a requirement.

A remote contact pad 126 is typically sized much larger than a conductive lead element 122, or electrode 118, and thereby provides enhanced structure operable to establish a connection for electrical communication between a source 130 and an electrode 118. In currently preferred embodiments, chip 102 is structured, by way of remote contact pads 126, to couple with an interrogation platform (not illustrated) that provides some, most, or substantially all control electronics. In certain preferred embodiments, an exposed surface of pad 126 is sized at least an order of magnitude larger than a surface area of one of electrodes 118 (in microchannel 106) to facilitate connecting device 102 to a remote electrical signal source 130. Desirably, contact pads 126 are configured and arranged to permit forming an electrical connection with a probe element, such as a spring-loaded pin, or with other cooperating electrically conductive structure.

Microchannel 106 provides fluid communication between a receiving chamber 134 and a holding chamber 138. Sometimes, one or more filter arrangement, generally indicated at 142 in FIG. 1, may be included in-circuit to resist passage of particulate material having a size too large for flow through microchannel 106. Chambers 134, 138 depicted in FIG. 1 are illustrated as being substantially circular in plan form, although in general, any shape factor is operable. A workable chamber 134, 138 should simply accommodate a volume of fluid. Typically, chambers 134, 138 are carried on chip 102. However, one or more of such chambers 134, 138 may be disposed remote from a chip 102. It may be convenient, e.g., in the case of potentially hazardous samples, for the chambers to be integral with chip 102 to facilitate containment of fluids to resist spread of pathogens, or other harmful substances.

The cross-section of microchannel 106 can have any desired size or configuration operable to cause a desired fluid flow arrangement. For example, the cross-section of a microchannel 106 in a blood analyzing MEMS chip 102 used to analyze blood cells may range in cross-sectional area from 35 to 250 μm². Naturally, other applications may require a larger or smaller area. For example, the invention may be used to identify and characterize a genetic specimen based on electrical properties, thus channel 106 may be of a size sufficiently small to require proteins to flow single file. Typically, when used as a particle counter detector, a chip 102 will have a microchannel 106 that is sized in cross-section to promote single-file flow, of the particles being counted, past the detecting electrodes. In addition, the channel may be substantially rectangular in cross section as illustrated, or it may also be a different shape, such as circular, semicircular, elliptical, or have a “V” shape or stepped “V.”

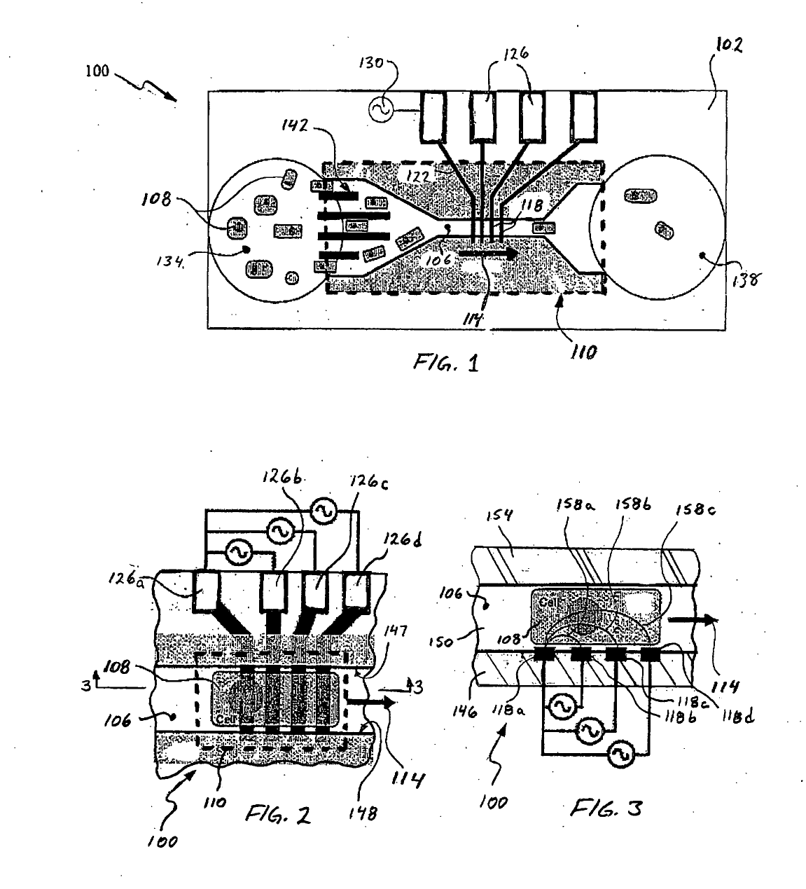

With reference to FIGS. 2 and 3, microchannel 106 is carried on a substrate 146. Desirably, substrate 146 is electrically non-conductive. Operable substrates 146 nonexclusively include plastics, such as polycarbonate, silicon, and glass. One operable substrate includes glass wafers available from Corning under the identifier “Pyrex 7740.” The manufacturing process generally includes pre-cleaning the substrates, for example, by way of a sulfuric acid/hydrogen peroxide solution, such as with a produce known as “Piranha etch.”

In certain preferred embodiments, walls 147, 148 of microchannel 106 are formed in a photoresist layer 150. An operable photoresist material includes an epoxy-based photoresist available under the commercial name “SU-8” from Microlithography Corp. of Newton, Mass. A photoresist layer 150 is typically between about 4-20 μm in thickness, depending upon the nature and size of particles to be interrogated using the MEMS chip.

In general, electrodes are made from electrically conductive materials, such as metals including gold, platinum, aluminum, copper, titanium, and the like. Alloys, and layered combinations of metals are also operable to form electrodes 118. Surface electrodes may be sputtered on to a thickness of about 1500 Angstroms, or so. An electrode pattern advantageously may be arranged using contact lithography, such as by using an EV 420 available from Electronics Visions, of Phoenix, Ariz. Traditionally, a positive photoresist (e.g., Shipley 1830 from Shipley Chemical, MA) is spun onto a surface of the substrate, then exposed developed and wet etched. Electrodes having greater thickness are typically electroplated onto the thus-formed foundation provided by surface electrodes.

A top, or cover layer 154 desirably is provided as a gasket to form microchannel 106 as a substantially fluid-tight conduit. Tops 154 can be made from an assortment of materials, nonexclusively including glass, silicone, and plastics or plastic-like materials, as well as laminates, such as film laminates. One desirable top 154 is made from polydimethylsiloxane. A gasket, such as a channel-sealing gasket made from silicone-based PDMS can be interposed between a top layer and a photoresist layer. An operable top can be made from a glass coverslip of the type used in preparation of slides for optical microscopic examination. Certain desirable top layers 154 are sufficiently transparent, in combination with the substrate, to permit optical monitoring of particles (e.g., by transmission light microscopy) for positioning and guidance of particles in the microchannel 106.

In certain embodiments, fluid-contacting (wetted) surfaces of the MEMS chip are coated with an anti-surface-binding protein such as heparin, or a di-block co-polymer (such as PS-PEO) to resist adsorbing, or adhering between the wetted surfaces of the MEMS chip 102 and the particles 108, or to resist cellular adhesion to the surface of the chip 102. Similarly, reagents and/or diluents may be provided in one or more chambers carried on, or placed in fluid communication with, structure carried on a MEMS chip.

A series of columns may be configured in photoresist layer 150 to form a filter 142 at the entrance to microchannel 106, and may further act to support the top surface 154. Desirably, filter 142 is configured to prevent larger debris from entering the microchannel 106 and clogging the detection device 100.

As illustrated in FIG. 1, microelectrodes 118 fully extend across the bottom of microchannel 106, thus creating an axially oriented detection zone between surface electrodes 118a-d (see, FIG. 3). Of course, electrodes 118 may have alternative arrangements, including being disposed in pairs transversely with respect to microchannel 106, or partially extending into the microchannel 106. The electrical signals between electrodes follow different paths 158a-c, depending on factors including the signal frequency, electrode configuration and spacing, and structure interposed between the electrodes. The presence of a particle 108, such as a cell, passing through the interrogation zone 110 modifies the overall device impedance. Depending on the interrogation frequency used, a percentage of the electrical signal is passed directly through the cell 108, and the resulting overall device impedance is directly influenced by the cellular dielectric properties and the whole cell shape and size.

Different electrical interrogation signals can be used in the invention to help distinguish between cell types, structure and state. In the most simple configuration, the electric impedance is measured between pairs (plus reference) of electrodes using an interrogation signal constructed using a single sinusoid, or complex broadband wave form, from 100 Hz-50 MHz. Presence of the cell in the channel alters the impedance between the electrodes in a way that is dependent upon the dielectric properties of cell constituents and upon the morphological structure. Individual cells can be identified by single or multi-frequency impedance spectra. In one envisioned use, voltages and currents between electrodes are used to from a spatial “image” of the cell to augment the temporal signature. In such, the system would generate spatial images of the electrical properties of cells at subcellular resolution—images that would change over time as the electrical properties of the cell membranes and/or constituents change with stimuli. In one aspect of the invention, the cellular membrane can be characterized using a higher radio frequency (approximately 1-50 MHz). The cytoplasm and other cell media can be characterized using a low frequency stimulus (approximately 1 kHz) which is dominated by the resistive shunt path around the cell. Further, because white blood cells (WBC) red blood cells (RBC) and platelets can each be characterized by the dielectric properties of its cellular media as well as the dielectric properties of its cellular membrane, and their spatial distribution, devices structured according to the invention provide an effective way to characterize major components of whole blood based on the electrical properties of its cellular structure. Furthermore, different interrogating temporal waveforms, spatial patterns and amplitudes can be used, depending on the specimen to be analyzed.

To achieve the desired result of characterizing blood cells based on their electrical impedance properties, the user simply places a specimen sample (e.g., a whole blood aliquot) into the fluid reservoir 134, and pumps it through the microchannel 106 and into the counted cell reservoir 138 in the flow direction 114 while applying and measuring appropriate electrical signals across the electrodes 118. The cells 108 can be pumped at any suitable rate for analysis, i.e., of about 0.5 to 10 μL/min., or so, using a commercially available pump, vacuum source, pressure reservoir, or other apparatus effective to cause controlled fluid flow.

FIG. 2 illustrates in greater detail the configuration of the detection zone 110 with a cell 108 therein. The multiple electrode detector 100, in combination with multiple radio-frequency analysis, enhances cell differentiation capabilities (e.g., fully developed systems are able to perform CBC analyses with a five-part white cell differential). The multiple electrode/multiple frequency (ME/MF) technique provides electrical impedance data dominated by the characteristics of the electrical shunt path around the cell, thereby providing cell size information at low frequencies (i.e., about 1-50 kHz). The higher frequencies (1-10 MHz) used in the ME/MF technique sends current through the cell membrane (penetrating the cell) at varying depths proportional to the electrode spacing as depicted in FIG. 3.

FIG. 4 illustrates a plan view of a MEMS chip 162 having an alternative configuration disposing eight electrodes 118 in the interrogation zone 110. Individual electrodes 118 are in electrical communication with individual remote contact pads 126a-h. An optional electrode 166 may be provided to communicate an electrical signal from remote pad 126i to the receiving chamber 138. Similarly, an optional electrode 170 may be provided disposed to communicate an electric signal from remote contact pad 126j to fluids in the receiving chamber 134.

A currently preferred electrode configuration may be characterized as a surface electrode. Such a surface electrode typically is formed substantially as a coating on a surface of the substrate 146 (FIG. 3), and forms a portion of the bottom surface of a microchannel 106. Surface electrodes typically are disposed to provide an electric signal in an axial direction with respect to the microchannel 106. However, it is within contemplation for surface electrodes to be disposed for substantially transverse electrical communication with respect to a microchannel 106.

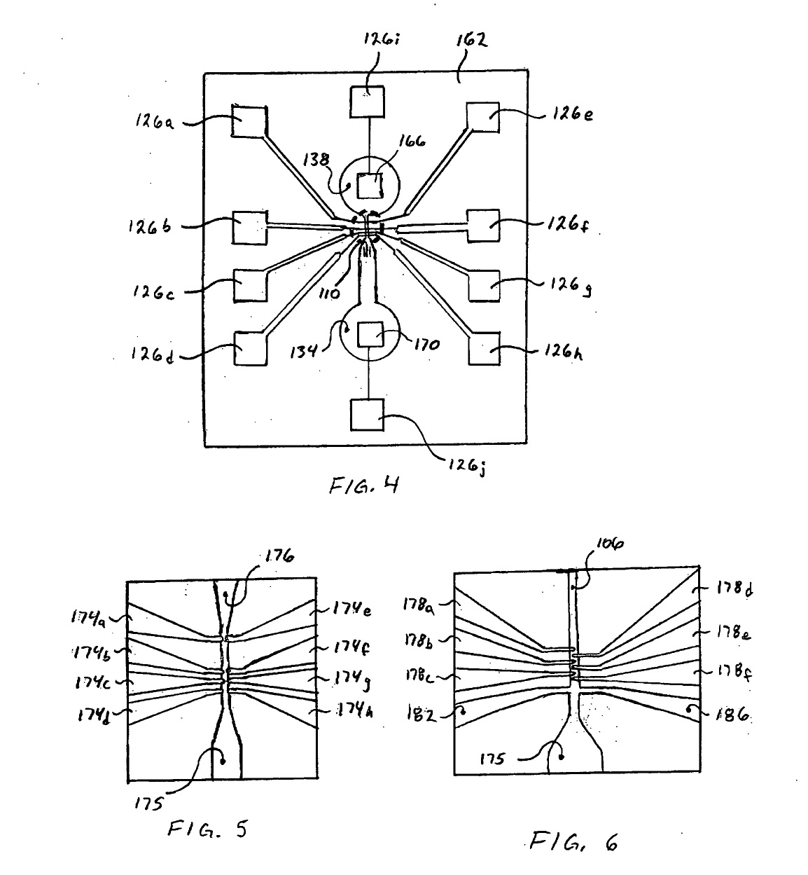

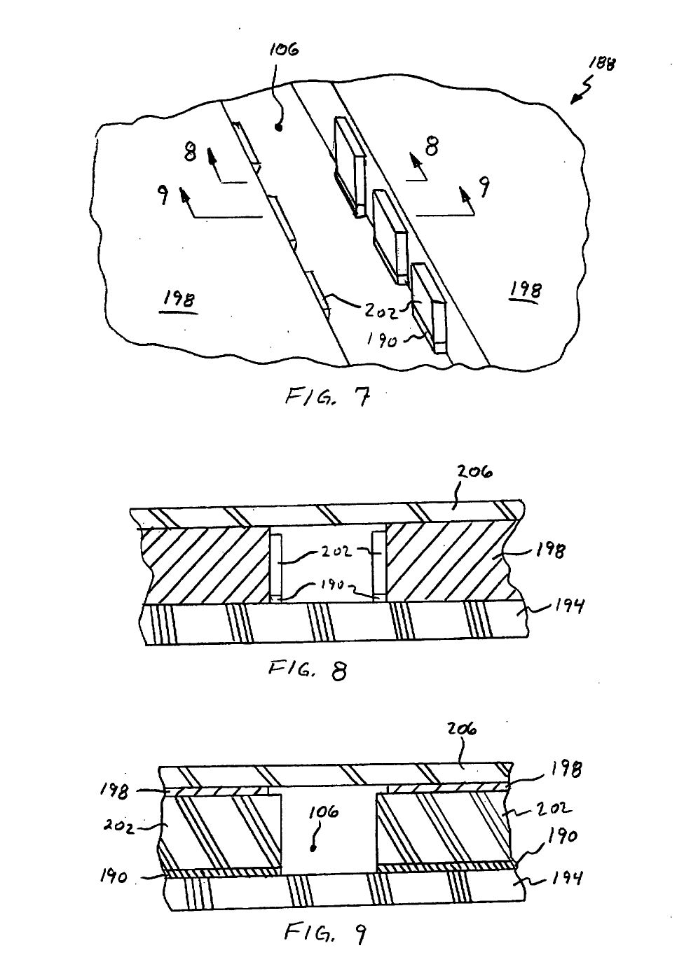

Electrodes 118 may be disposed in the interrogation zone 110 in any desired axially spaced apart pattern. Furthermore, electrodes 118 may be configured as desired to dispose a wetted surface for transmitting or receiving an electrical signal into, across, or along the microchannel 106. For example, electrodes 118 may be disposed in pairs to communicate an electric signal therebetween substantially transverse to the microchannel 106. Transversely disposed electrodes typically, although not necessarily, are configured to occupy a portion of a wall of the microchannel 106. FIG. 5 illustrates an arrangement of electrodes 174a-h that are transversely disposed with respect to the microchannel 106 extending between entrance 175 and exit 176. Also, or in an alternative configuration, electrodes 118 may be arranged to transmit an electrical signal in an axial direction with respect to the microchannel 106. FIG. 6 illustrates an arrangement of six electrodes 178a-f that are axially disposed with respect to the microchannel 106. Sometimes, it may be useful to include one or more fluid delivery conduits 182, 186, e.g., to convey drugs or insertion components to a poration target area in a microchannel 106.

MEMS chips 102, 162, etc., can be manufactured using any known method operable to form the desired structure. For example, a detector MEMS chip 102 may be micromachined using any number of techniques, including CMOS, or modified CMOS techniques. It is currently preferred to manufacture certain MEMS chips using lithographic techniques.

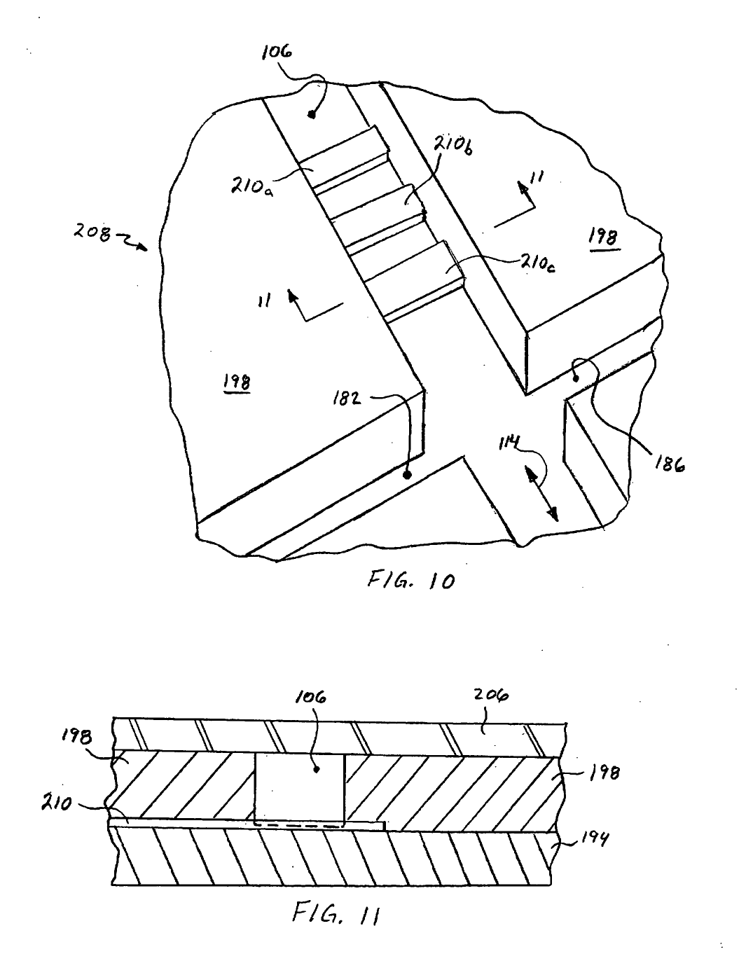

The following process was used to manufacture a MEMS chip 188 having substantially full-thickness electrodes as illustrated in FIGS. 7-9: a metal seed layer 190 (250 Å Ti and 750 Å Au) was sputtered on three-inch diameter quartz glass (Pyrex 7740) substrates 194, patterned and etched to form the approximate electrode dimensions, electrical connecting pads, and the conducting grid for electroplating. An approximately 4.5 μm thick layer of epoxy-based photoresist 198 (e.g., SU-8, available from Microlithography Chemical Corp., Newton, Mass.), was spun over the metal seed layer and patterned to form the fluid reservoirs and the wider portions of the connecting channel. Gold microelectrodes 202 were slowly electroplated using a low current density (1 mA/cm²) until the electrodes were about 0.5 μm below the top surface of the photoresist. Following electroplating, the photoresist was cured on a hot plate at 125° C. for 15 minutes to reduce cracking during subsequent fabrication procedures. A 6000 Å thick layer of aluminum was then sputtered on the entire wafer, patterned, and etched to act as a mask for the microchannel etching procedure. An oxygen plasma etch was used to form the narrowest portion of the microchannel 106 (˜10 μm wide), which lies between the gold electrodes 202. (It is also within contemplation to form electrodes 202 on only one side wall, or bottom wall, forming the microchannel 106.) The unwanted photoresist and aluminum were then wet etched from the surface of the wafer and thoroughly rinsed in DI water in preparation for bonding of the coverslip 206. A coverslip, or the top layer 206, typically is shaped to avoid blocking access to remote contact pads 126, and generally provides access for fluid transfer to receiving reservoir 134, and permits access to holding chamber 138, as required, e.g., to promote fluid flow.

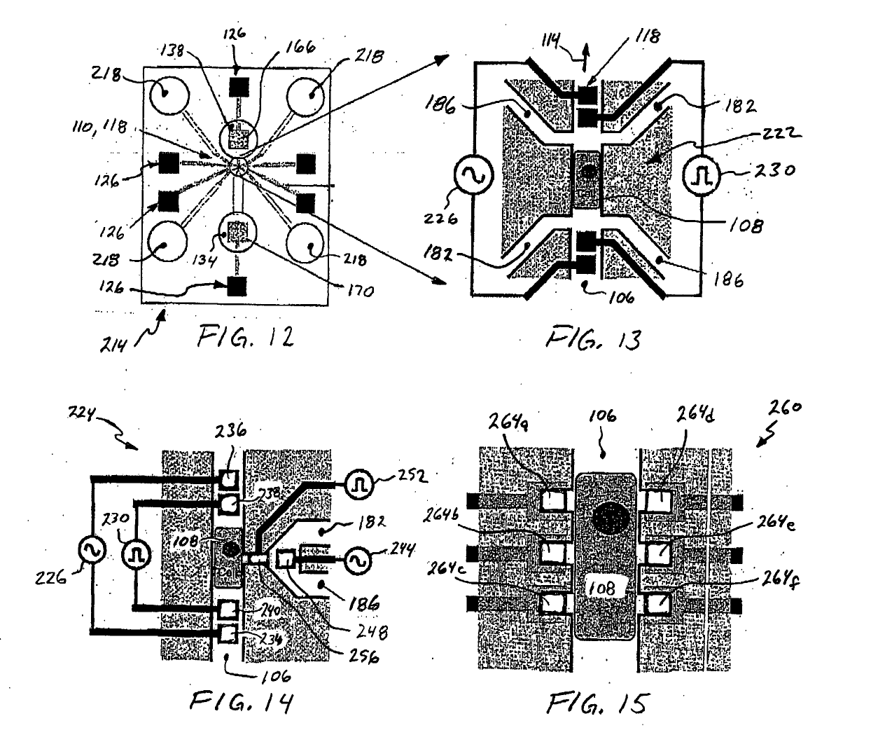

FIGS. 10 and 11 illustrate certain alternative details of construction of a MEMS chip 208 within the ambit of the invention. MEMS chip 208 includes a plurality of surface electrodes 210a-c. While three such electrodes 210 are shown, additional electrodes 210 may also be included in a chip 208. It is currently preferred to provide electrodes arranged in groupings of three to five. Sometimes, a first grouping of two or more electrodes 210 may be disposed axially spaced apart along microchannel 106 from a second grouping of electrodes 210. Additionally, any desired spacing may be arranged between two or more electrodes 210. Often, a spacing between two adjacent electrodes is on the order of, and desirably is less than, a cell length of a cell to be interrogated. An electrode spacing of less than a cell length desirably permits resolving electrical properties of different regions of the cell. Further, if desired, one of more of fluid delivery conduits 182, 186 may also be included in a chip 208. Such fluid delivery conduits advantageously may be arranged to place one or more reservoirs holding a treatment fluid into fluid communication with microchannel 106.

FIGS. 12 and 13 illustrate a currently preferred structural arrangement for a MEMS chip, generally indicated at 214, operable to perform electroporation activities on selected cells 108. As illustrated in FIG. 12, chip 214 carries a plurality of electrodes 118 disposed to transmit an electric signal into microchannel 106 in an interrogation zone 110. Optionally, one or more of electrodes 166, 170 may be included to permit transmission or reception of an electric signal with respect to holding chamber 138 and receiving chamber 134, respectively. One or more treatment fluid chambers 218 is carried on chip 214, and is placed into fluid communication with channel 106 through an associated fluid delivery conduit, such as one of conduits 182, 186. Chambers 218 may be loaded with a fluidized treatment substance during manufacture of a chip 214, or may be loaded prior to use of the chip 214. A fluid delivery conduit desirably introduces the treatment fluid into a portion of microchannel 106 that may be regarded as an electroporation treatment zone, generally indicated at 222 in FIG. 13.

With reference to FIG. 13, an indicating signal 226 may be applied between a pair of microelectrodes 118. An electroporation signal 230 may be applied to a second pair of electrodes 118. One or more electrodes associated with the indicating signal and the electroporation signal can be used in common. It is further within contemplation that such signals could be applied to the same pair of electrodes. Desirably, the pair of electrodes used for the electroporation signal is disposed downstream from at least one of the indicating signal electrodes. Such a downstream configuration can provide a feedback signal for a control system to trigger, or to apply, the electroporation signal.

FIG. 14 illustrates an alternative arrangement operable to perform electroporation activities on cells 108, and generally indicated at 224. An indicating signal 226 is applied between a pair of electrodes, such as electrodes 234 and 236. An electroporation signal 230 is applied between a pair of electrodes, such as electrodes 238 and 240. One or more fluid delivery conduits 182, 186 provides delivery of a treatment substance into a treatment zone in microchannel 106. A conduit 182 or 186 may be used to hold a cell 108 in position by application therethrough of a slight negative pressure in addition to delivering macromolecules directly to the cell 108 for electro-injection. A second indicating signal 244 can be applied between electrode 248 and one of the other electrodes on the chip. The second signal 244 can be effective to indicate passage of treatment substance, such as a macromolecule. A second electroporation signal 252 can be applied, if desired, between electrode 256 and a second electrode carried on the chip structure 224. Electroporation signal 252 may be used to modify a treatment substance, or to further modify porosity of a cell 108. In certain cases, electroporation signal 252 may be provided as the sole electroporation signal to produce an alternative effect on cells 108, for example, to produce alternative pore structure in a cell 108.

FIG. 14 illustrates structure useful in a wide variety of electroporation/micro-injection applications. The structural arrangement 224 may be used to advantage in quantitative (e.g., number of molecules injected per cell) fluorescence-based electro-injection studies; to hold cells in position via light suction; and to investigate controlling intracellular voltages via induced long-duration membrane pores or chemically created pores. Furthermore, the structure 224 may be employed in whole-cell patch clamp operations. Devices including structure 224 are operable to gain access to an interior of a cell 108 via long-term dielectric breakdown (electroporation) of a small area of the cell's membrane and/or by adding a chemical such as nystatin through a conduit 182, 186.

FIG. 15 illustrates alternative structure, generally indicated at 260, useful in certain electroporation activities. Structure 260 includes recessed surface electrodes 264a-f. Such recessed surface electrodes 264 substantially eliminate direct metal contact on the surface of a cell 108, increasing biocompatibility. Desirably, the surface electrodes 264 are made from platinum to reduce impedance at the electrode/electrolyte interface. In certain cases, electrodes 264 may be formed as double-layer electrodes by plating with platinum black to further reduce impedance at the electrode/electrolyte interface. The six-electrode grouping of electrodes 264a-f enables transverse or lateral control of membrane potentials (electroporation) between selected pairs of electrodes 264a, 264c, 264d, and 264f with simultaneous electric impedance interrogation on the middle electrodes 264b and 264e. As illustrated in FIG. 15, in certain electroporation and/or certain cell detection activities, a grouping of electrodes 264a-c or 264d-f desirably is spaced apart to occupy a length of microchannel 106 on the order of, or smaller than, a length of a cell 108.

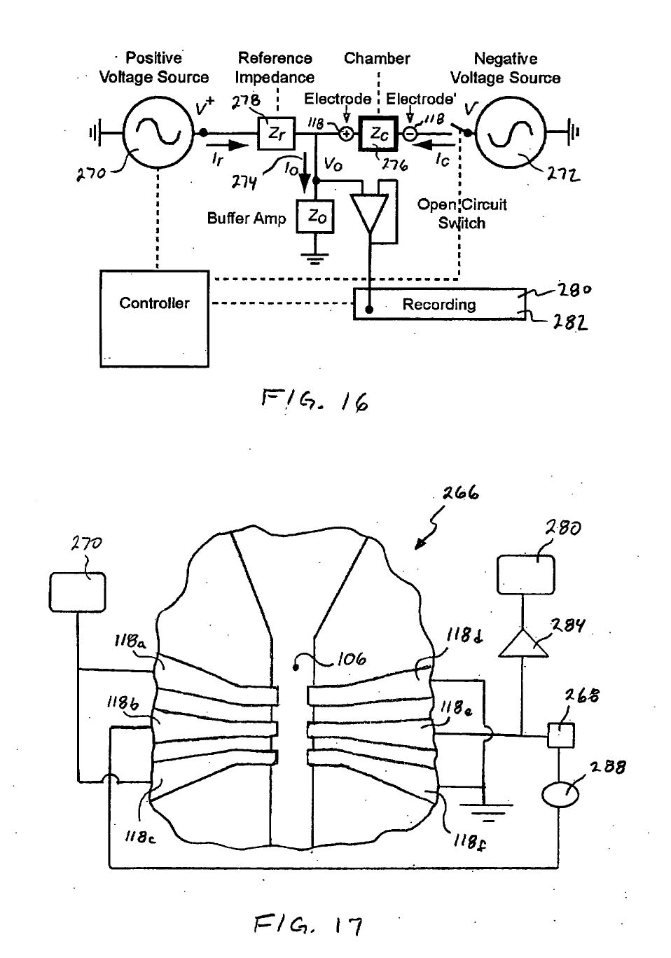

Control circuitry used to operate MEMS chips structured according to the instant invention typically includes field-effect transistor (FET) operational amplifiers, waveform generators, lock-in amplifiers, and reference impedances. FIG. 16 illustrates an equivalent electrical circuit corresponding to one control circuit useful in controlling membrane injection and for voltage clamping. Waveform generator 270 is driven 180 degrees out-of-phase from waveform generator 272. A pair of signals introduced by generators 270, and 272 may be broadcast into microchannel 106 by way of selected pairs of electrodes 118 to direct the transmitted signals either along an axis of microchannel 106, transverse to such axis, or in some way into or through the microchannel. A cooperating pair of electrodes 118 is then selected to measure the corresponding impedance in the microchannel 106. The system can be calibrated on a frequency-by-frequency basis to account for buffer amp leak current. A deviation or imbalance between chamber impedance and reference impedances correlates to changes caused by presence of cellular material in the vicinity of the electrodes effective to perturb local impedance. The imbalance may be measured and recorded using a lock in amplifier 280, and a digital oscilloscope 282.

FIG. 17 illustrates one operable circuit arrangement, generally indicated at 266, operable as a voltage-divider electrical control arrangement for using the invention as a cell detector. An operational amplifier circuit with the unknown impedance in the feedback leg is one alternative method. In one realization, the control electronics 266 allow for independent addressing of the electrodes (e.g., 118a-f) for frequency-domain electric impedance measurements and/or patterning of the extracellular electric field in microchannel 106. The control electronics 266 include a serial, instrumented reference impedance 268 that is used for current measurement. The reference impedance 268 and configured MEMS chip is calibrated using open-circuit and shunt measurements. In some cases, an unused electrode pair is used as a saline-bridge reference. The impedance interrogation has been validated to exceed (substantially) the accuracy level of commercial network analysis systems over the range 100 Hz-40 MHz. Impedance interrogation signals were provided using a multi-channel, differential, arbitrary waveform generator 272 (e.g., Tektornix AWG430). Signals were recorded via computer controlled quadrature lock-in amplifiers 280 (e.g., Stanford Research SR830 and 844). Calibration was done on a frequency-by-frequency basis before and after the experiment, although this could also be done in the time domain or using Fourier methods. Due to the relatively high frequencies involved, it was important for the calibration to account for the impedance of the onboard FET amplifiers 284 and the phase delays between various sampling points. This was accomplished via a series of open-circuit and short-circuit calibrations using the relevant pairs of electrodes. The calibration procedure was validated by measuring the impedance of samples having known dielectric properties. Time domain signals, when needed, were recorded using a digital oscilloscope. Quadrature outputs were recorded using GPIB and/or a 16 bit, 333 kHz analog to digital converter (National Instruments). The entire system was controlled by computer (Apple Macintosh or PC) via GPIB (EIEE488, National Inst) and DAC (National Instruments) using the programming environment Igor Pro (Wave Metrics).

The overall electric impedance, Z(w), between any specific pair of electrodes on the μ-EI device was calculated from the lock-in data. The raw magnitude and phase data reflect the dielectric properties of the interconnecting spring-loaded pins, micro-device geometry, and any interactions with the cell/media in the recording zone. To characterize the MEMS device and the electrode/electrolyte interface, we measured Z(w) after filling the channel with extracellular media. Impedance records taken in the absence of cells (the control condition) were used to characterize the current-density dependent electrode-electrolyte impedance as well as the impedance of the interface (shunt impedance around the chamber plus access impedance). The interface impedance was the same during the control vs. cell-loaded condition and therefore, if desired, could easily be removed from the raw impedance results—but this step is not necessary for simple CBC sorting and clustering analysis.

A waveform generator 270 is applied across electrodes 118a and 118d, and 118c and 118f to a ground reference. Lock in amplifier 28 is connected across electrodes 118b and 118e through FET op amp 284. Reference impedance 268 is calibrated, as well as shunt impedance 288. Changes measured by lock in amplifier 280 correlate to changes in impedance caused by presence of cellular matter near the electrodes 118b and 118e (in the interrogation zone). It is to be realized that the electrical circuit examples illustrated in FIGS. 16 and 17 are illustrative only, and are not to be regarded as the only operable configurations. Many variations may be made to form equally operable alternative control circuitry.

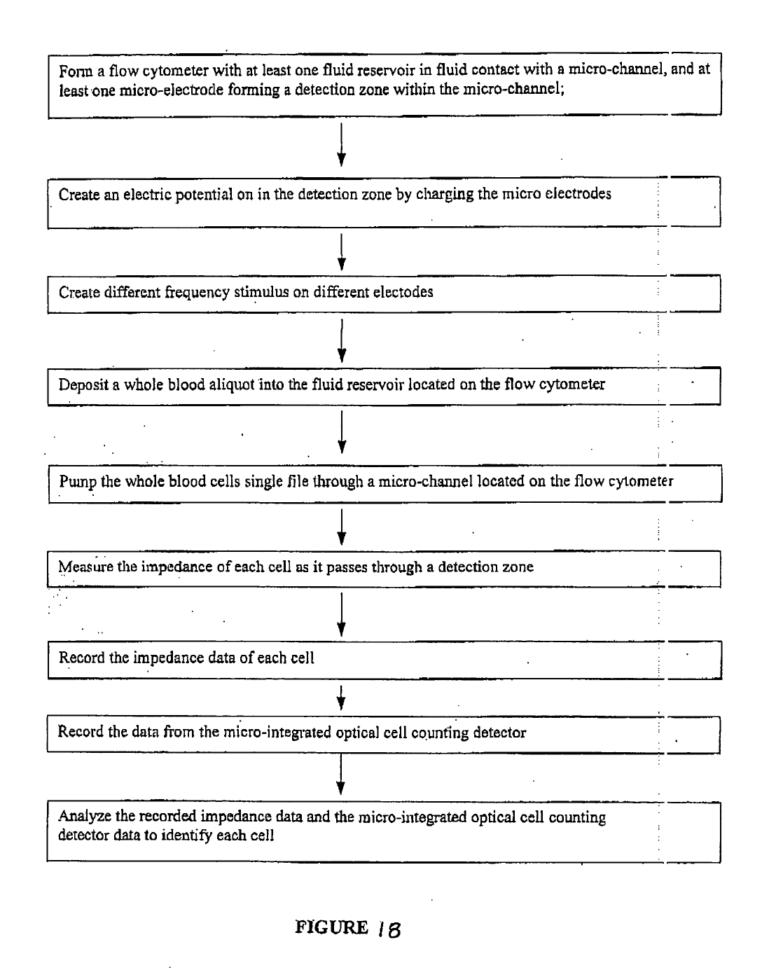

Devices structured according to the invention can be used as cell counting devices using electric impedance (e.g., for CBC tests, sperm counts, dairy milk testing, etc.). An example of one method for using a detector structured according to principles of the invention in a calibration exercise for subsequent use of the detector device as a hematology analyzer is presented and described in FIG. 18. Of course, certain illustrated steps may be modified, repeated, or even skipped, in uses within the ambit of the instant invention. Steps may further be performed in an order other than illustrated, as desired to accomplish a certain result.

In general, the invention may be practiced using a flow cytometer including a MEMS chip carrying structure such as illustrated in any of FIGS. 1 through 15. Structure of the MEMS chip, such as a size of cross-section through a microchannel 106 and/or an arrangement of microelectrodes 118, will generally be scaled or patterned according to the size of the particles to be interrogated. A sample, including the particles carried in a fluid, is introduced into the receiving chamber. The sample may be treated with one or more reagent, such as an anti-clotting factor or a diluent, prior to placement in the receiving chamber. Alternatively, such reagent may be added to the receiving chamber during manufacture of the MEMS chip, or prior to use of the chip. Some sort of motive force is then applied to urge the sample (fluid and particles) to flow through the microchannel and an interrogation zone toward the holding chamber. A vacuum applied to the holding chamber, or pumping arrangements may be employed to cause such fluid flow. Valve arrangements may be formed to permit control of fluid flow from the receiving chamber, and/or one or more other chamber(s), through the microchannel.

As the fluid and particles are moving through the microchannel, an electric signal is imposed by a signal generator between a pair or more of microelectrodes disposed in the microchannel. A wide variety of electric control circuits of different configurations are operable with devices constructed according to the instant invention, depending upon the desired result. It is within contemplation for portions of control circuitry (e.g., solid state integrated circuit devices) to be formed on a chip, or carried on a chip, in addition to external circuit elements.

For example, a relatively simple circuit is required to enable use of certain MEMS chips as particle indicators or cell counters. In other applications, additional circuit elements are included in the control circuitry, for example, op. amps, oscilloscopes, and feedback loops, for electroporation activities. Additionally, various hardware, such as an optical microscope, may be included in certain systems within contemplation to include a user's visual feedback in a process. The formed electrical circuit may encompass selected electrodes disposed to transmit, and/or measure, an electrical signal that may be characterized as being oriented transverse to the microchannel, aligned with an axis of the microchannel, or at an angle with respect to the channel axis.

Typically, the impedance between one or more pairs of electrodes is measured, and may be recorded and/or used in further data processing and/or for in-process feedback, as the fluid and particles pass through the interrogation zone. Changes in measured impedance, and/or the actual measured values, may be correlated to known changes and values corresponding to parameters associated with different particles to differentiate between types of particles in the sample, and/or to determine a number of particles passing through the interrogation zone.

In another aspect, the invention can be used as a micro-domain voltage clamp. This use especially benefits the research market (or scientific community) and provides a new platform for cellular electro-physiology experiments. Using the invention, the voltage potential of cell membranes can be regionally controlled using a non-invasive (no patch electrodes) technique. The electrical properties of neurons, for example, can now be studied in a controlled environment (i.e., drugs can be introduced to the cell during electrical interrogation).

In yet another aspect, the invention can be used in single cell electroporation for drug delivery/gene therapy. The invention may be used to deliver gene vectors or drugs to cells (via electroporation) as the cells flow through the electroporation zone. A treatment substance may be added to the sample directly, or as the sample is loaded into the receiving chamber. Also, fluid containing a treatment product (such as a drug, or gene, etc.) can be introduced to one or more treatment fluid chambers disposed in fluid communication with a treatment zone in the microchannel 106. In the latter case, the treatment fluid is urged toward the treatment zone. Individual cells may be retained in a treatment position by a suction applied through a conduit in communication with the treatment zone in a microchannel 106, or by use of voltage clamping. Electroporation using embodiments having surface electrodes reduces structural damage to cells flowing through the microchannel by reducing contact damage imparted by the electrodes. The electrical control circuit may be adapted to sense the presence of a cell in the electroporation zone in a feedback control loop. In any case, a relatively large voltage spike is applied across the cell membrane. Small holes (pores) can be formed in the cell membrane responsive to the voltage spike, and the drug, treatment product, or DNA can then diffuse into the cell.

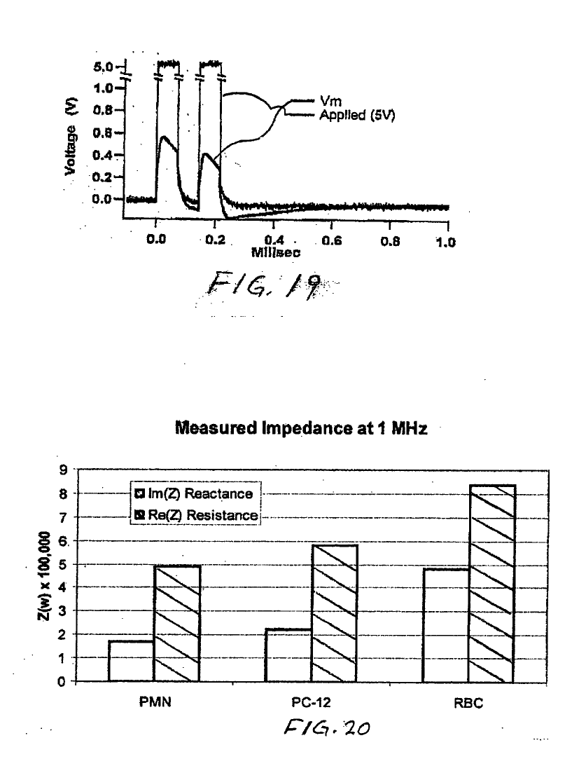

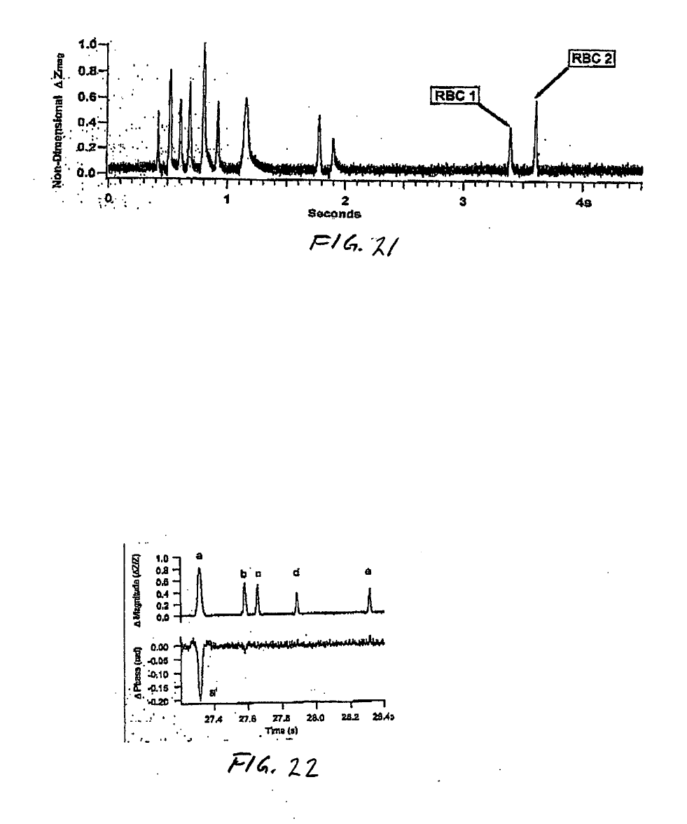

FIGS. 19 through 22 illustrate data obtainable in use of certain MEMS chips constructed according to the invention. Data presented in FIGS. 19-22 were collected using a MEMS chip structured and disposed in a control circuit as illustrated in FIG. 17, and where the electrodes 118a-f were substantially full thickness (channel depth) and transversely disposed across the microchannel 106. FIG. 19 shows an applied square-wave voltage signal 290, and a corresponding passive response 292. FIG. 20 illustrates the measured reactance and resistance recorded for three cell types. FIG. 21 illustrates the change in measured impedance corresponding to passage of a number of cells through an interrogation zone in a microchannel 106. FIG. 21 illustrates a difference in measured phase and magnitude of impedance corresponding to different cell types. The data identified with indicia a and a′ in FIG. 21 correspond to a white blood cell, and the data identified with b, c, d, and e correspond to passage of red blood cells through a detection zone between instrumented electrodes.

Having described these aspects of the invention, it is understood that the invention defined by the appended claims is not to be limited by particular details set forth in the above description, as many apparent variations thereof are possible without departing from the spirit or scope thereof.

1. A microelectrical-mechanical system (MEMS) device comprising:
a substantially electrically non-conductive substrate;
a machinable layer carried on said substrate;
a microchannel formed in said machinable layer and disposed in fluid communication between said receiving chamber and said holding chamber effective to transport a fluid stream, a portion of said microchannel being sized in cross-section to effect substantially single-file flow of said particles through an interrogation zone having a length;
a plurality of electrodes carried on said substrate and disposed in association with said zone operably to transmit an electrical signal into said microchannel;
a plurality of remote contact pads, separate ones of said contact pads being in electrical communication with separate ones of said electrodes and comprising a contact surface sized at least an order of magnitude larger than a surface area of its associated electrode to facilitate connecting said device to a remote electrical signal source; and
a cover layer disposed to form a substantially fluid-tight top to said microchannel.

2. The MEMS device of claim 1, wherein:
said electrodes are disposed on a single wall of said microchannel and spaced apart along an axis of said microchannel to form an electrical signal therebetween oriented substantially along said axis.

3. The MEMS device of claim 1, wherein:
said interrogation zone occupies a portion of said microchannel having a cross-section of substantially uniform size along said length; and
a plurality of said electrodes are disposed spaced apart in a direction oriented parallel to said length; and
a boundary wall of said microchannel is arranged spaced apart opposite one electrode by a distance sized in general agreement with a size of a particle to be interrogated effective to cause said particle to approach substantially into contact with said one electrode.

4. The MEMS device of claim 3, wherein:
a spacing between said boundary wall and said one electrode is about 10 μm.

5. The MEMS device of claim 1, wherein:
a cross-section of said microchannel is sized between about 20 μm² and about 250 μm².

6. The MEMS device of claim 1, further comprising a filter disposed in association with an entrance to said microchannel.

7. The MEMS device of claim 6, wherein:
said filter is formed in said machinable layer.

8. The MEMS device of claim 1, further comprising:
a fluid delivery conduit disposed to provide fluid communication between a treatment zone in said microchannel and a treatment fluid chamber.

9. The MEMS device of claim 8, wherein:
said treatment fluid chamber is carried on said substrate.

10. The MEMS device of claim 1, wherein:
a first pair of electrodes is operably arranged to detect the presence of a cell in said microchannel;
a second pair of electrodes is arranged downstream of at least an individual one of said first pair of electrodes effective to deliver an electroporation signal to said cell in an electroporation treatment zone.

11. The MEMS device of claim 10, further comprising:
a fluid delivery conduit disposed to provide fluid communication between a treatment fluid chamber and said electroporation treatment zone.

12. The MEMS device of claim 11, further comprising:
an electrode arrangement in association with said fluid delivery conduit and operable to detect passage of treatment substance therethrough.

13. The MEMS device of claim 11, further comprising:
an electrode arrangement in association with said fluid delivery conduit and operable to apply an electroporation signal in the vicinity of said electroporation treatment zone.

14. The MEMS device of claim 11, further comprising:
a conduit disposed to provide fluid communication between said electroporation treatment zone and a vacuum source operable to resist motion of a cell from said electroporation treatment zone.

15. The MEMS device of claim 1, further comprising:
an integrated electrical circuit component carried on said substrate and disposed in-circuit with at least one electrode.

16. The MEMS device of claim 1, further comprising:
a coating applied to wetted surfaces of said microchannel operable to resist build-up of biologic material in said microchannel.

17. The MEMS device of claim 1, wherein:
said electrodes comprise surface electrodes disposed on a bottom surface of said microchannel, and are spaced apart along an axis of said microchannel.

18. The MEMS device of claim 1, wherein:
said substrate and said cover layer are sufficiently transparent in combination to permit transmission of light therethrough to permit optical surveillance of cell motion through said microchannel.

19. A method for performing microanalysis of particles using a microelectrical-mechanical system (MEMS) chip to electrically interrogate the particles, said method comprising:
providing a said MEMS chip comprising a microchannel disposed in fluid communication between a receiving chamber and a holding chamber, said microchannel being structured directly to urge said particles into substantially single-file flow past a plurality of electrodes disposed in a detection zone within said microchannel;
loading a sample comprising particles entrained in a fluid into said receiving chamber;
applying a motive force operable to urge flow of a portion of said sample through said microchannel in a direction toward said holding chamber;
measuring a state of electrical impedance between a pair of said electrodes, while said portion is flowing through said microchannel, to obtain impedance data from the detection zone; and
performing analysis of said impedance data to determine the presence of one of said particles in said detection zone.

20. The method according to claim 19, further comprising:
performing analysis of said impedance data to determine one or more physical characteristics of certain of said particles.

21. The method according to claim 19, further comprising:
applying an electroporation signal between a pair of said electrodes effective to enhance permeability of a membrane of at least certain of said particles subsequent to detection of one of said particles in said detection zone.

22. The method according to claim 21, further comprising:
applying a restraining force on one of said particles effective to resist movement of said one particle from a treatment zone in said microchannel.

23. The method according to claim 22, further comprising:
urging flow of a treatment fluid, comprising a treatment substance, from a treatment fluid chamber through a conduit disposed in fluid communication with said treatment zone effective to introduce said treatment substance to the vicinity of said one particle.

24. The method according to claim 19, further comprising:
performing analysis of the impedance data to determine an instantaneous flow rate through said microchannel based upon time-of-flight of one of said particles between a first detected position and a second detected position disposed downstream of said first detected position.

25. A method for performing microanalysis of particles contained in a whole blood sample using a microelectrical-mechanical system (MEMS) chip to electrically interrogate the particles, said method comprising:
providing a said MEMS chip comprising a microchannel disposed in fluid communication between a receiving chamber and a holding chamber, said microchannel being structured directly to urge said particles into substantially single-file flow past a plurality of electrodes disposed in a detection zone within said microchannel, and comprising a boundary wall disposed spaced about 10 μm apart from one of said electrodes;
adding anti-coagulant to said sample;
loading said sample into said receiving chamber;
applying a pressure differential to said sample operable to urge flow of a portion of said sample through said microchannel in a direction toward said holding chamber;
measuring and recording a state of electrical impedance between a pair of said electrodes, while said portion is flowing through said microchannel, to obtain impedance data from the detection zone; and
performing analysis of said impedance data to determine the presence of one of said particles in said detection zone.
